(19) United States
(12) Patent Application Publication
(10) Pub. No.: US 2025/0140388 A1
(43) Pub. Date: May 1, 2025

(54) NUCLEAR MEDICINE SCAN SCHEDULER

(71) Applicant: Siemens Medical Solutions USA, Inc., Malvern, PA (US)

(72) Inventors: Abhinav Shanker, Bengaluru (IN); Noby Varghese, Bangalore (IN)

(21) Appl. No.: 18/682,461

(22) PCT Filed: Feb. 28, 2023
(86) PCT No.: PCT/US2023/014040

§ 371 Date: Feb. 9, 2024

(51) Int. Cl. G16H 40/20; A61B 5/055; G16H 10/60; G16H 50/70

(52) U.S. Cl. CPC G16H 40/20; A61B 5/055; G16H 10/60; G16H 50/70

(57) ABSTRACT

A framework for scheduling nuclear medicine scanning. The framework may search a database for a patient record of a previous patient that is most similar to a current patient based on first patient and scan attributes associated with the current patient. The patient record of the most similar patient includes second patient and scan attributes. In response to the first scan procedure associated with the current patient matching the second scan procedure of the patient record, a scan duration may be predicted based on the patient record to generate a predicted scan duration for the current patient.

### TECHNICAL FIELD

The present disclosure generally relates to data processing, and more particularly, to planning a schedule for nuclear medicine scanning of a patient.

### BACKGROUND

Nuclear medicine scanning is a method of producing medical images of a patient by detecting radiation emitted by a radioactive tracer (or radiopharmaceuticals) from different parts of the body after the radioactive tracer is administered to the patient. Medical image data is then acquired and reconstructed by a medical scanner (e.g., positron emission tomography or PET scanner) and reviewed by a nuclear medicine physician, who interprets the image data to make a diagnosis.

The physician typically schedules the patient to undergo a nuclear medicine scanning procedure for a particular date and time. The most common ways to schedule nuclear medicine procedures are via verbal and written communications. However, many issues can complicate scheduling. For example, dose delivery to the facility may be disrupted. Additionally, the medical scanner may be shut down due technical glitches or mandatory system maintenance, resulting in the need for rescheduling of scans.

These problems make it very difficult to plan for patient scans and assess how many patients can be scanned on a given day. Inefficient scheduling may lead to either wastage of scanner time or doses of radiopharmaceuticals.

### SUMMARY

Described herein is a framework for scheduling nuclear medicine scanning. In accordance with one aspect, the framework searches a database for a patient record of a previous patient that is most similar to a current patient based on first patient and scan attributes associated with the current patient. The patient record of the most similar patient includes second patient and scan attributes. In response to the first scan procedure associated with the current patient matching the second scan procedure of the patient record, a scan duration may be predicted based on the patient record to generate a predicted scan duration for the current patient.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 is a block diagram illustrating an exemplary system;

FIG. 2 is a block diagram illustrating an exemplary medical facility;

FIG. 3 shows an exemplary method of scheduling nuclear medicine scanning; and

FIG. 4 shows an exemplary guidance table for adjusting the scan duration extracted from a similar patient record.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed; on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance.

Unless stated otherwise as apparent from the following discussion, it will be appreciated that terms such as “segmenting,” “generating,” “registering,” “determining,” “aligning,” “positioning,” “processing,” “computing,” “selecting,” “estimating,” “detecting,” “tracking,” “deriving,” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods can be compiled for execution on a variety of hardware platforms and interface to a variety of operating systems. In addition, implementations of the present framework are not described with reference to any particular programming language. It will be appreciated that a variety of programming languages may be used.

While scheduling a nuclear medicine scan, a myriad of factors needs to be meticulously considered. Identifying the patient type is an imperative factor because every patient may need different care. The scan time for each patient may differ based not only on the protocol used, but also on the patient's physical and disease conditions. The availability of each medical scanner also needs to be tracked. Currently, the physician or technician uses experience to estimate the overall scan duration. Based on the estimated scan duration, administration of radiopharmaceuticals for the next patients is planned so that they are ready for their scans.

The present framework facilitates nuclear medicine scan scheduling. In some implementations, different attributes that impact scan duration (e.g., patient mobility state) are collated and used to predict the overall utilization of a medical scanner (i.e., scan durations) for future patients. The scan durations may be used to automatically plan radiopharmaceuticals administration times and schedule of patients for scanning on respective scanners for a given day. The attributes used for scan duration prediction may include patient attributes, such as those entered during registration of the patient (e.g., scan procedure, height, weight), as well as scan attributes, including but not limited to, administered radiopharmaceuticals dose, residual activity, administration time, planned scan procedure, delay duration, medical scanner model, technician information, or a combination thereof. Such attributes may be automatically provided to the medical scanner module, thereby reducing manual entry error and increasing the effectiveness of dose management at the facility, since it presents the technician a clear overview of how much activity is leftover in the radiopharmaceutical vial for the next injections.

In some implementations, the scheduler generates alerts based on the protocol set by the user to ensure that delayed scans are not missed. This avoids manual tracking of the study by the technician. The scheduler plans the scans by considering any delays in patient study, as well as scanner downtime. When the medical scanner is down, the scheduler facilitates accommodation of patients administered with the radiopharmaceuticals and helps ensure that patients are quickly and efficiently scanned. The scheduler may alert the technician to properly plan the patient scan after considering preventive maintenance logs of the medical scanner. The scheduler may also determine the amount of radiopharmaceuticals to be procured by the hospital to scan the planned number of patients for a given day. These and other features and advantages will be described in more detail herein.

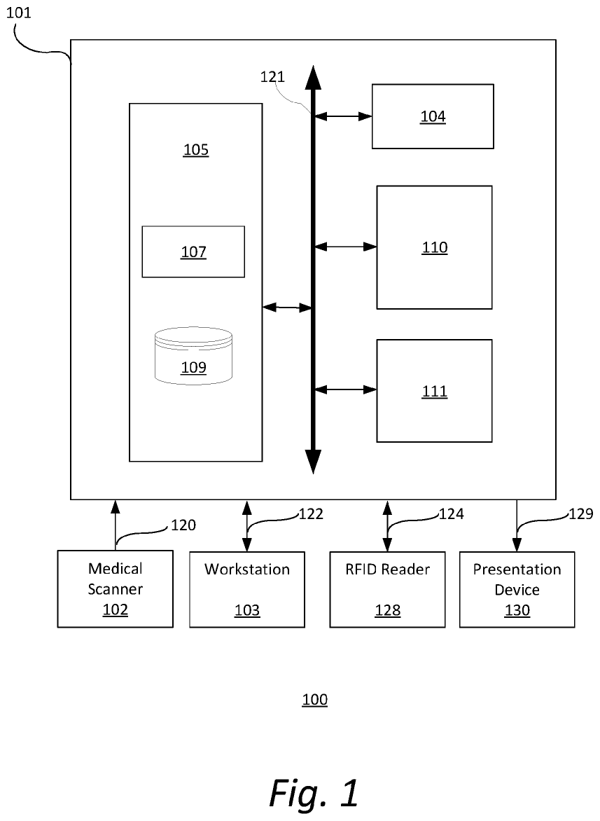

FIG. 1 is a block diagram illustrating an exemplary system 100. System 100 includes a computer system 101 for implementing the framework as described herein. In some implementations, computer system 101 operates as a standalone device. In other implementations, computer system 101 may be connected (e.g., using a network) to other machines, such as medical scanner 102, workstation 103, radio-frequency identification (RFID) reader 128 and presentation device 130. In a networked deployment, computer system 101 may operate in the capacity of a server (e.g., thin-client server), a cloud computing platform, a client user machine in server-client user network environment, or as a peer machine in a peer-to-peer (or distributed) network environment.

In some implementations, computer system 101 comprises a processor device or central processing unit (CPU) 104 coupled to one or more non-transitory computer-readable media 105 (e.g., computer storage or memory), a display device 110 (e.g., monitor) and various input devices 111 (e.g., mouse or keyboard) via an input-output interface 121. Computer system 101 may further include support circuits such as a cache, a power supply, clock circuits and a communications bus. Various other peripheral devices, such as additional data storage devices and printing devices, may also be connected to the computer system 101.

The present technology may be implemented in various forms of hardware, software, firmware, special purpose processors, or a combination thereof, either as part of the microinstruction code or as part of an application program or software product, or a combination thereof, which is executed via the operating system. In some implementations, the techniques described herein are implemented as computer-readable program code tangibly embodied in non-transitory computer-readable media 105. In particular, the present techniques may be implemented by a scheduler 107.

Non-transitory computer-readable media 105 may include random access memory (RAM), read-only memory (ROM), magnetic floppy disk, flash memory, and other types of memories, or a combination thereof. The computer-readable program code is executed by CPU 104 to process data retrieved from, for example, medical scanner 102, workstation 103, RFID reader 128 and database 109. As such, computer system 101 is a general-purpose computer system that becomes a specific-purpose computer system when executing the computer-readable program code. The computer-readable program code is not intended to be limited to any particular programming language and implementation thereof. It will be appreciated that a variety of programming languages and coding thereof may be used to implement the teachings of the disclosure contained herein.

The same or different computer-readable media 105 may be used for storing a database (or dataset) 109. Such data may also be stored in external storage or other memories. The external storage may be implemented using a database management system (DBMS) managed by the CPU 104 and residing on a memory, such as a hard disk, RAM, or removable media. The external storage may be implemented on one or more additional computer systems. For example, the external storage may include a data warehouse system residing on a separate computer system, a cloud platform or system, a picture archiving and communication system (PACS), or any other hospital, medical institution, medical office, testing facility, pharmacy or other medical patient record storage system.

Medical scanner 102 is a radiological scanning modality that acquires medical image data 120 associated with patients. Medical scanner 102 may acquire medical image data 120 using techniques such as, but not limited to, high-resolution computed tomography (HRCT), magnetic resonance (MR) scanning, computed tomography (CT), helical CT, X-ray, angiography, positron emission tomography (PET), fluoroscopy, single photon emission computed tomography (SPECT), or a combination thereof. Scanner information (e.g., installation date, maintenance cycle, periodic downtime) associated with the medical scanner 102 may be stored in database 109.

Workstation 103 may include a tablet, mobile device, laptop or computer intended to be used by the medical technician. Workstation 103 may include a graphical user interface to receive user input via an input device (e.g., keyboard, mouse, touch screen, voice or video recognition interface) to input medical data 122. Workstation 103 can be operated in conjunction with the entire system 100. For example, workstation 103 may communicate directly or indirectly with the medical scanner 102 and/or RFID reader 128 so that the medical image data 120 and/or unique identifier 124 are displayed at workstation 103. Workstation 103 may also provide or retrieve and display patient data, radiopharmaceuticals administration data (e.g., RA time, predicted and actual dose), scan data (e.g., scan duration, delayed scan time) of a given patient and/or scanner information (e.g., maintenance or downtime information). Multiple workstations 103 may be provided in a given facility.

RFID reader 128 is a device that has one or more antennas that emit radio waves and receive signals back from an RFID tag. The RFID tag may be an active or passive device that stores an identifier unique to a particular patient. The unique identifier may be used to look up and retrieve patient information from computer system 101.

Presentation device 130 may be any display device (e.g., monitor, television) or sound device (e.g., speaker) that is used to present schedule information 129 that is visible to the public. One or more presentation devices 130 may be located in, for example, the reception area, a radiopharmaceuticals administration (RA) room, a pre-scan room, a scanning room and/or the post-scan room. Schedule information 129 may include, but is not limited to, the number of patients in the queue, wait time, the respective patient's RA time, scanning time and/or delayed scanning time.

It is to be further understood that, because some of the constituent system components and method steps depicted in the accompanying figures can be implemented in software, the actual connections between the systems components (or the process steps) may differ depending upon the manner in which the present framework is programmed. Given the teachings provided herein, one of ordinary skill in the related art will be able to contemplate these and similar implementations or configurations of the present framework.

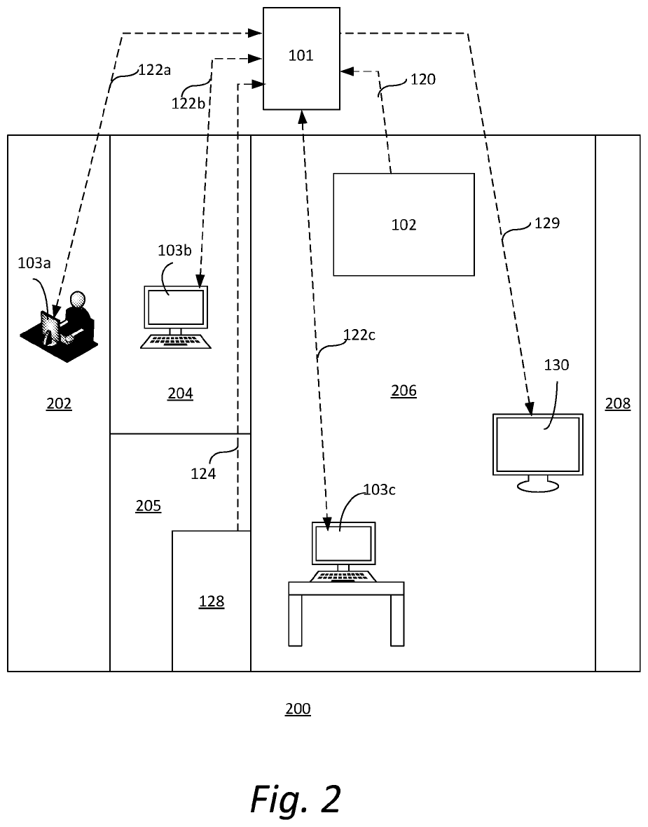

FIG. 2 is a block diagram illustrating an exemplary medical facility 200 where the exemplary system 100 may be deployed. Medical facility 200 includes a reception area 202, a radiopharmaceuticals administration (RA) room 204, a pre-scan room 205, a scanning room 206 and a post-scan room 208. It should be appreciated that the layout and number of rooms are shown for illustration purposes. Any other layout or rooms may also be applicable.

When a patient arrives at reception area 202, the receptionist registers the patient. The receptionist may enter or retrieve patient data 122a, including patient attributes, via workstation 103a. Patient data 122a is communicated to computer system 101. Patient attributes in patient data 122a may include, but are not limited to, age, gender, weight, height, requested scan procedure, mobility status, history of claustrophobia or anxiety disorder, etc. Other patient data 122a may include, but is not limited to, patient name, diagnosis, history of risks, known allergic reactions, ambulatory information, and so forth. Other types of patient data 122a may also be provided. The patient may be provided with an RFID tag that stores a unique identifier. If the patient is visiting for the first time, a unique identifier is generated for the new patient. Otherwise, the unique identifier is retrieved from computer system 101. The unique identifier may be used to look up and retrieve patient information from computer system 101.

The patient then enters the radiopharmaceuticals administration room 204. The blood glucose level of the patient may be checked to minimize dietary glucose-related competitive inhibition of the radiopharmaceuticals. Additionally, the patient's creatine level may also be checked. The blood glucose and creatine levels may be displayed on workstation 103b. If the blood glucose and creatine levels do not exceed predetermined threshold values, the patient is administered the radiopharmaceuticals. Various ways may be used to administer the radiopharmaceuticals. For example, the radiopharmaceuticals may be orally given by mouth, injected, or placed into the eye or bladder.

In some implementations, scheduler 107 estimates the dose of radiopharmaceuticals to be administered to the patient based on patient data 122a. Patient dose may be estimated based on, for example, type of scan, patient weight and amount of dose remaining at the facility. Scheduler 107 may keep track of the amount of dose used and the amount of dose ordered by the facility. When the remaining dose is less than what is required by the current patients, scheduler 107 may automatically split the remaining dose between the current patients based on a predetermined lower threshold. The predetermined lower threshold may be provided by, for example, the user during initial setup of scheduler 107. As an example, the hospital may typically administer a dose of 10 mCi. However, if the remaining dose in the hospital is only 25 mCi (e.g., due to decay of activity) and there are 3 current patients to be scanned, scheduler 107 may split the remaining dose to 10 mCi, 8 mCi and 7 mCi based on the patient weight and scan protocol. Alternatively, scheduler 107 may split the remaining dose more evenly among the 3 patients to 9 mCi, 8 mCi and 8 mCi based on patient weight and type of examination. After administration of the radiopharmaceuticals, the actual administration data 122b (e.g., method, time, dose, activity) may be entered at workstation 103b and sent to the computer system 101 via workstation 103b. The patient waits in the pre-scan room 205 for a period (e.g., 30-60 min) during which the uptake of radiopharmaceuticals occurs before scanning.

Prior to entering the scanning room 206, the RFID tag associated with the patient is scanned by RFID reader 128. RFID reader 128 sends the unique identifier 124 stored in the RFID tag to computer system 101, thereby notifying computer system 101 that the patient is entering scanning room 206. Patient data 122c is retrieved by workstation 103c based on the unique identifier 124. The patient is loaded onto a scanning table of the medical scanner 102. After the patient is loaded, the patient is scanned by medical scanner 102.

In some instances, one or more delayed scans are required as part of a scan protocol, where the patient needs to be scanned multiple times (e.g., two or three times). For example, some nuclear scan protocols require the patient to be scanned after radiopharmaceuticals administration (i.e., baseline scan), and then scanned again after a delay duration of one or two hours (i.e., delayed scan) to determine how the activity of the radiopharmaceuticals is progressing in the patient's body. If a delayed scan is determined to be required by the scan protocol, a recommended delayed scan time is derived from a delay duration and reserved for the patient on the scanning schedule. The delay duration (e.g., 1 hour) may be defined by the technician and stored in database 109 as a scan protocol setting. If the delayed scan is performed later than the recommended delayed scan time, the quality of image and interpretation may be impacted.

The patient may wait in the post-scan room 208 during the delay duration until an alert is generated prior to the patient's turn to indicate that the patient may enter the scanning room 206 and the scan may begin shortly (e.g., 15 min). The alert may include, for example, a sound and/or light flashing on workstation 103c, a light emitting diode (LED) blinking on the patient's RFID tag, or a combination thereof. Scheduler 107 may also alert the technician (e.g., by displaying a message on workstation 103c) to avoid planning additional patients to be scanned during the delay duration, so that the same medical scanner is available for the delayed scan of the current patient.

If the delay duration is greater than the predicted scan duration, computer system 101 may identify the next patient from the schedule. The identified patient's name and any delayed scan information 129 may be retrieved from computer system 101 and presented on presentation device 130. The technician may inform the next patient in the pre-scan room 205 of the delay.

After the scan is completed, a medical image may be reconstructed and reviewed for image quality. The patient is unloaded from the table and waits in the post-scan room 208. The patient may wait in the post-scan room 208 until the radioactivity is reduced to an acceptable level. The actual scan duration may be recorded and communicated to computer system 101 via workstation 103c. The “scan duration” (or scan time) as used herein generally refers to the amount of time the patient occupies the scanning room 206. The scan duration may be defined by the time interval between the patient entering scanning room 206 (e.g., swipe-in using RFID tag) and the patient exiting scanning room 206 (e.g., swipe-out using RFID tag) and entering the post-scan room 208. In some cases, when the patient is scheduled to undergo a delayed scan, the scan duration may be defined by the first time duration when the patient is in the scanning room 206, the time duration when the patient waits in the post-scan room 208, the second time duration when the patient is in the scanning room 206 for the delayed scan. Once scanning room 206 is vacated by the current patient, the next scheduled patient can enter the scanning room 206.

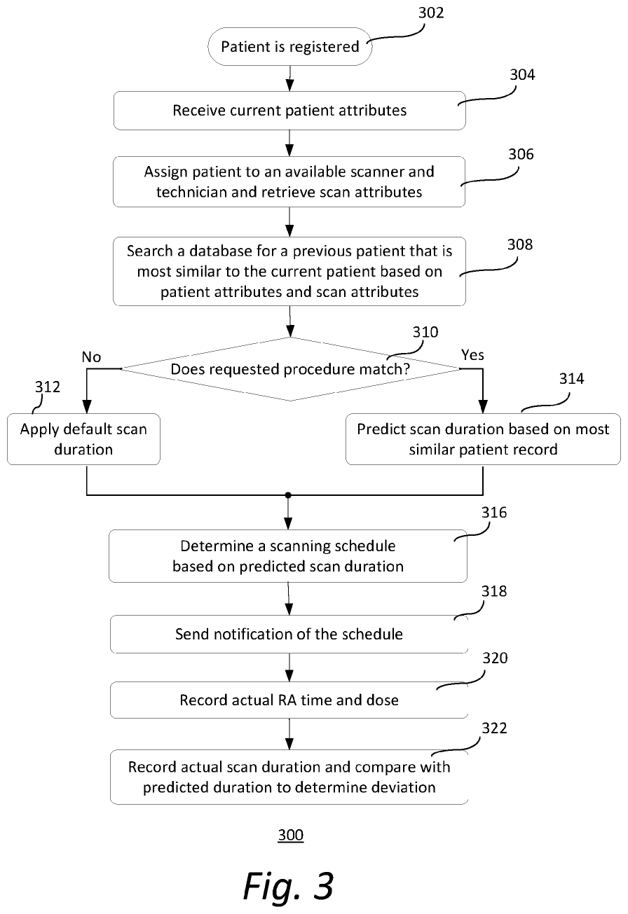

FIG. 3 shows an exemplary method 300 of scheduling nuclear medicine scanning. It should be understood that the steps of the method 300 may be performed in the order shown or a different order. Additional, different, or fewer steps may also be provided. Further, the method 300 may be implemented with the system 100 of FIG. 1, medical facility 200 of FIG. 2, a different system or facility, or a combination thereof.

At 302, the method 300 starts when a current patient is registered.

At 304, scheduler 107 receives patient attributes of the current patient. Patient attributes may be entered and/or retrieved by workstation 103a at the reception area 202. Patient attributes may include, but are not limited to, age, gender, weight, height, requested scan procedure, mobility status, history of claustrophobia or anxiety disorder, etc.

At 306, scheduler 107 assigns the current patient to a medical scanner and technician and retrieves scan attributes associated with the assigned medical scanner and technician. The assignment may be performed based on the availabilities of the medical scanner and technician. Additionally, if the patient had been scanned earlier at the same facility and is undergoing a follow-up or delayed scan after an initial baseline scan, the patient is assigned to the same medical scanner used for the baseline scan. In the case of a delayed scan, scheduler 107 may block the time on the scanning schedule and remind the technician by generating an alert, so that no other patient may be assigned to the same medical scanner during the delay duration. Scheduler 107 may also use the scan attributes associated with the baseline scan to determine the delay duration and dose for the follow-up or delayed scan.

Once the patient is assigned to a medical scanner, scheduler 107 can look up scan attributes associated with the medical scanner. Scan attributes associated with the assigned medical scanner may be retrieved from database 109 and presented on, for example, workstations 103a and 103b in the reception area 202 and RA room 204 respectively. Such scan attributes may include, but are not limited to, the assigned medical scanner model and information of the technician (e.g., name, experience) performing the scan. The retrieved scan attributes associated with the assigned medical scanner may be used to determine how long it typically takes for the technician to run the scan on the assigned medical scanner. Additionally, scheduler 107 may retrieve scan attributes associated with the patient (e.g., delay duration, administered dose) in the baseline scan to propose the dose and scanning delay for the follow-up scan for better comparability.

At 308, scheduler 107 searches database 109 for a patient record of a previous patient that is most similar to the current patient based on the patient attributes and scan attributes. The goal is to find a similar patient with the greatest number of matching patient and scan attributes, as these patient and scan attributes may impact the scan duration. The assumption is that if all the patient and scan attributes match between the current and previous patient associated with the patient record, the scan durations for these two patients should be similar.

Database 109 may store patient records of previously scanned patients who have visited the medical facility 200. Each patient record stores one or more patient attributes and one or more scan attributes associated with a previous patient. Database 109 may initially include default scan duration values. As more and more patients are scanned, database 109 collects more and more patient records that provide more data points for upcoming new patients. With more data points, the probability of finding a match between the new patient and previously scanned patients increases.

At 310, scheduler 107 compares the requested scan procedure of the current patient with the scan procedure description of the most similar patient record to determine if they match.

If there is no match, at 312, scheduler 107 applies the default scan duration as the predicted scan duration. Since the scan procedures do not match, the remaining patient and scan attributes of the similar patient record do not need to be checked since the scan duration in the similar patient record is not applicable to the current patient. The default scan duration may be retrieved from, for example, database 109. The default scan duration indicates a typical scan duration of the requested scan procedure plus a predetermined adjustment time period. The default scan duration may be dependent on the facility, and defined by the staff of the facility when the scheduler 107 is first installed at the facility.

If there is a match, at 314, scheduler 107 predicts the scan duration based on the most similar patient record. If the patient and scan attributes of the similar patient record exactly match the current patient's attributes, the scheduler 107 applies the scan duration in the similar patient record as the predicted scan duration. For example, if the patient mobility status, age, weight, prior history of claustrophobia or anxiety disorder, scanning delay and technician experience in the similar patient record match those attributes of the current patient, the actual scan duration in the similar patient record is applied as the predicted scan duration. In some implementations, if the actual scan duration is substantially different from the predicted scan duration in the similar patient record, scheduler 107 may apply the average of the actual and predicted scan durations of the similar patient record as the predicted scan duration of the current patient.

Otherwise, the non-matching patient and scan attributes for the current and previous patients may be used to adjust the actual scan duration extracted from the similar patient record to generate the predicted scan duration. The comparison may be performed in a predetermined priority order. The extracted scan duration may be adjusted based on, for example, patient mobility status, age, weight, prior history of claustrophobia, scanning delay and technician experience and/or other attributes extracted from the similar patient record. Each attribute may be assigned a weight in accordance with the priority to adjust the scan duration.

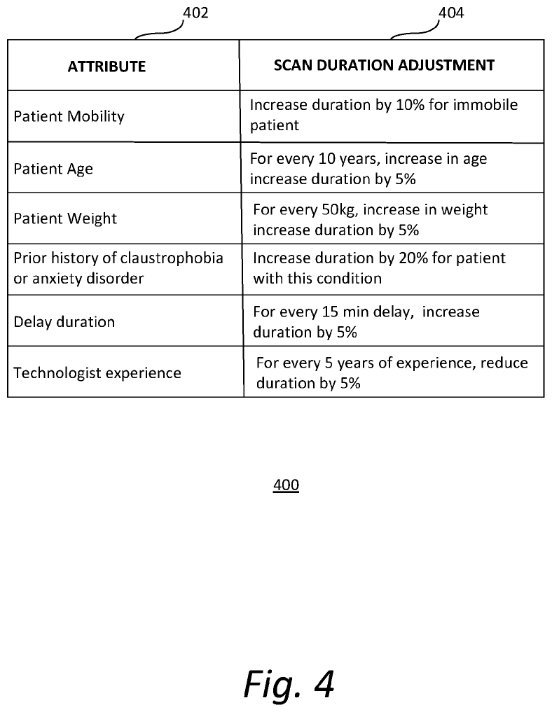

FIG. 4 shows an exemplary guidance table 400 for adjusting the scan duration extracted from a similar patient record. Column 402 shows a patient or scan attribute (e.g., patient mobility, age, weight, prior history of claustrophobia/anxiety disorder, delay duration, technician experience) and column 404 shows the corresponding adjustment (e.g., increase or decrease by a predetermined percentage amount) of the scan duration. It should be appreciated that these adjustments are shown for illustration purposes, and other types of adjustments based on other types of attributes may also be useful. For example, the scan duration may be adjusted based on the model of the medical scanner, since newer models may scan faster than older models. These adjustments may be the starting points. As the database collects more patient records, machine learning algorithms may be applied to determine these adjustments.

In an exemplary case, the current patient A is a 50-year-old person in a wheelchair who has requested a bone scan. The technician assigned to perform the scan is an expert with 10 years of experience and is performing the scan on the latest scanner. Scheduler 107 searches the database 109 for the most similar patient record associated with a previous patient B. If all the attributes match between patient A and patient B, scheduler 107 simply uses the previous scan duration in the most similar patient record as the predicted scan duration for patient A. However, if only the patient age, weight and technician assigned to patient B matches those attributes of patient A, and patient B is mobile (i.e., not on a wheelchair), the previous scan duration for patient B may be increased by 10% to generate the predicted scan duration for patient A.

Returning to FIG. 3, at 316, scheduler 107 determines a scanning schedule based on the predicted scan duration. The dose of radiopharmaceuticals to be administered to the current patient may also be derived based on the predicted scan duration. The scanning schedule may include reservations for the current patient for radiopharmaceuticals administration (RA) and scanning (e.g., baseline, follow-up and/or delayed scans). The RA time may be reserved on the scanning schedule based on the current number of patients in the queue for scanning, the predicted scan durations of the queued patients and any scanning delay for the current patient. In cases where a delayed scan is required by the scan protocol, a buffer time (or delay duration) may be inserted to the predicted time duration to ensure that the scan of the previous patient is completed within any stipulated scanning delay of the current patient. The buffer time may be, for example, a predetermined duration (e.g., 10-20%).

In an exemplary scenario, Patient X is the current patient, and Patients A and B are already in the queue for scanning. Patient A is scheduled by scheduler 107 to be scanned at 10:00 AM, while Patient B is scheduled by scheduler 107 to be scanned at 10:30 AM. It is expected that Patient B′s scan will be completed by around 11:10 AM. Scheduler 107 back-calculates and determines that Patient X needs an imaging delay of 60 minutes, and therefore should be injected at 10:10 AM so that he can be scanned at 11:10 AM.

The technician may choose to provide an additional buffer in the start phases. For example, since Patient X is injected at 10:10 AM, he should be scanned by 11:30 AM latest, since the activity of the radiopharmaceutical (considering Fluorodeoxyglucose or FDG in this case) may significantly impact image quality after this time. The technician may insert a buffer time for Patient B and indicate that Patient B may take longer. Patient X should only be injected at 10:20 AM, allowing a 10-minute buffer for the scan duration of Patient B.

The derived RA time and scan times may be used to plan the schedule for the current patient, the patients in the queue as well as the incoming new patients for the day to optimize use of the scanning resources (e.g., scanning room, medical scanner, doses available at the facility). In addition to reducing manual error, automation of scheduling advantageously produces more efficient and accurate results.

At 318, scheduler 107 sends notification of the derived RA time and the scan time. For example, the scheduler 107 sends a notification to the workstation 103b in the RA room 204, workstation 103c and presentation device 130 in the scanning room 206 to display the derived RA time and the scan time for the current patient. Workstation 103b may inform the technician of the RA time and the dose for the current patient. The patient may then receive the dose of radiopharmaceuticals at or around the recommended RA time.

At 320, scheduler 107 records the actual RA time and dose in the current patient's record after the administration of the radiopharmaceuticals. When the patient enters the scanning room 206, the patient's record may be displayed at workstation 103c.

At 322, scheduler 107 records the actual scan duration after the scanning of the current patient is completed and the patient exits the scanning room 206. The recording of the actual scan duration may be initiated in response to, for example, the scanning of the RFID tag of the current patient upon exit of the scanning room 206. It should be appreciated that other mechanisms of automatically initiating the recording of the entry and/or exit of the patient from the scanning room 206 are also useful. The actual scan duration is stored in database 109 for future comparisons. As more patient records are added to the database 109, the probability of finding a matching patient record for future new patients increases.

Scheduler 107 may compare the actual scan duration with the predicted scan duration to determine any deviation in prediction. A trained neural network non-linear regression method may be used to determine the percentage deviation in prediction. The percentage deviation may be used to determine the adjustments to the scan duration, thereby increasing the accuracy of prediction.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. One or more non-transitory computer readable media embodying a program of instructions executable by machine to perform operations for scheduling nuclear medicine scanning, the operations comprising:
receiving one or more first patient attributes and one or more first scan attributes associated with a current patient, wherein at least one of the one or more first patient attributes comprise a first scan procedure;
searching a database for a patient record of a previous patient that is most similar to the current patient based on the one or more first patient attributes and the one or more first scan attributes, wherein the patient record comprises one or more second patient attributes and one or more second scan attributes;
comparing the first scan procedure with a second scan procedure in the patient record;
in response to the first scan procedure matching the second scan procedure, predicting a scan duration based on the patient record to generate a predicted scan duration for the current patient; and
determining a scanning schedule based on the predicted scan duration.

2. The one or more non-transitory computer readable media of claim 1, wherein the operations further comprise:
in response to the first scan procedure not matching the second scan procedure, applying a default scan duration as the predicted scan duration for the current patient.

3. The one or more non-transitory computer readable media of claim 1 wherein the one or more first and second patient attributes comprise age, gender, weight, height, mobility status, history of claustrophobia or anxiety disorder, or a combination thereof.

4. The one or more non-transitory computer readable media of claim 1 wherein the first and second one or more scan attributes comprise administered radiopharmaceuticals dose, residual activity, administration time, planned scan procedure, delay duration, medical scanner model, technician information, or a combination thereof.

5. The one or more non-transitory computer readable media of claim 1 wherein predicting the scan duration based on the patient record to generate the predicted scan duration comprises:
in response to the one or more first patient attributes and the one or more first scan attributes associated with the current patient matching the one or more second patient attributes and the one or more second scan attributes in the patient record, predicting the scan duration based on an actual scan duration in the patient record.

6. The one or more non-transitory computer readable media of claim 1 wherein predicting the scan duration based on the patient record to generate the predicted scan duration comprises:
in response to at least one of the one or more first patient attributes or at least one of the one or more first scan attributes associated with the current patient not matching the at least one of the one or more second patient attributes and at least one of the one or more second scan attributes in the patient record, adjusting an actual scan duration in the patient record based on the at least one of the one or more first and second patient attributes or the at least one of the one or more first and second scan attributes to generate the predicted scan duration.

7. The one or more non-transitory computer readable media of claim 1 wherein determining the scanning schedule comprises reserving a radiopharmaceuticals administration time and a scan time on the scanning schedule for the current patient.

8. The one or more non-transitory computer readable media of claim 7 wherein determining the scanning schedule further comprises reserving a delayed scan time on the scanning schedule for the current patient.

9. The one or more non-transitory computer readable media of claim 8 wherein the operations further comprise:
generating an alert to remind the current patient to enter a scanning room at the delayed scan time.

10. The one or more non-transitory computer readable media of claim 9 wherein the alert is generated on a radio frequency identification (RFID) tag.

11. The one or more non-transitory computer readable media of claim 1 wherein the operations further comprise:
recording an actual scan duration in response to scanning of an RFID tag.

12. A method of scheduling nuclear medicine scanning, comprising:
receiving one or more first patient attributes and one or more first scan attributes associated with a current patient, wherein at least one of the one or more first patient attributes comprises a first scan procedure;
searching a database for a patient record of a previous patient that is most similar to the current patient based on the one or more first patient attributes and the one or more first scan attributes, wherein the patient record comprises one or more second patient attributes and one or more second scan attributes;
comparing the first scan procedure with a second scan procedure in the patient record; and
in response to the first scan procedure matching the second scan procedure, predicting a scan duration based on the patient record to generate a predicted scan duration for the current patient.

13. The method of claim 12 further comprising estimating a dose of radiopharmaceuticals to be administered to the current patient based on the one or more first patient attributes and an amount of dose remaining at a facility.

14. The method of claim 12 further comprising determining a delayed scan is required after a delay duration.

15. The method of claim 14 further comprising generating a first alert to remind the current patient to enter a scanning room for the delayed scan.

16. The method of claim 15 wherein the first alert is generated on a radio frequency identification (RFID) tag.

17. The method of claim 14 further comprising generating a second alert to remind a technician to avoid planning additional patients to be scanned during the delay duration at a same medical scanner used for the current patient.

18. The method of claim 14 further comprising applying scan attributes associated with a baseline scan to the delayed scan.

19. The method of claim 12 wherein predicting the scan duration based on the patient record to generate the predicted scan duration comprises:
in response to the one or more first patient attributes and the one or more first scan attributes associated with the current patient matching the one or more second patient attributes and the one or more second scan attributes in the patient record, applying an actual scan duration in the patient record as the predicted scan duration for the current patient.

20. A scheduling system, comprising:
a non-transitory memory device for storing computer readable program code; and
a processor device in communication with the non-transitory memory device, the processor device being operative with the computer readable program code to perform steps including
receiving one or more first patient attributes and one or more first scan attributes associated with a current patient, wherein at least one of the one or more first patient attributes comprises a first scan procedure;
searching a database for a patient record of a previous patient that is most similar to the current patient based on the one or more first patient attributes and the one or more first scan attributes, wherein the patient record comprises one or more second patient attributes and one or more second scan attributes;
comparing the first scan procedure with a second scan procedure in the patient record; and
in response to the first scan procedure matching the second scan procedure, predicting a scan duration based on the patient record to generate a predicted scan duration for the current patient.
